(19) United States
(12) Patent Application Publication
(10) Pub. No.: US 2022/0240992 A1
(43) Pub. Date: Aug. 4, 2022

(54) Temporary sternal stabilization device to prevent right ventricular rupture

(71) Applicant: Vineet Mehan, Great Falls, VA (US)

(72) Inventor: Vineet Mehan, Great Falls, VA (US)

(21) Appl. No.: 17/162,603

(22) Filed: Jan. 29, 2021

(51) Int. Cl. A61B 17/82

(52) U.S. Cl. CPC A61B 17/823

(57) ABSTRACT

The invention relates to a sternal closure device for temporarily closing and stabilizing a sternum in between surgeries of the thoracic cavity wherein a C-shaped hook attaches to the outside of the portion of one side of a dissected sternum which is connected to an opposing C-shaped hook by a wire which may be readily cut with surgical scissors to allow physicians quick access to the thoracic cavity for surgical treatment. The device includes a stress measurement dial that allows physicians to evaluate the force needed for permanent closure fixation. The new and unique aspect of this invention is that it gives surgeons easy access to the thoracic cavity without worrying about the sternal bones fusing together between operations.

### CROSS REFERENCE TO RELATED APPLICATION

This application claims the benefit of U.S. Provisional Patent No. 62/995,535, filed Jan. 31, 2020, which is incorporated by reference herein in its entirety.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

The present invention relates to a system for longitudinal closure of dissected sternums including holding together two portions of a dissected sternum, such as resulting from a sternotomy.

The sternum, along with the rib cage, serves to protect vital internal organs, such as the heart, from damage. In cases where access to a vital organ is needed, a longitudinal incision is typically made along a midline of the sternum and the two resulting sides of the sternum are then separated to provide the surgeon access to the patient's thoracic cavity.

Once the procedure is completed, the two portions of the sternum are held together and stabilized to protect the internal organs of the thoracic cavity from exterior forces as well as from damage caused by friction between edges of the dissected sternum against internal organs. Some patients require multiple surgeries to the cavity to perform multiple surgical washouts, or debridements, to remove tissue that has been damaged by infection or be otherwise unviable, such as in a deep sternal wound infection. Some patients require multiple surgeries within the cavity in a short period of time, therefore, a permanent system of closing the dissected portions of the sternum is inappropriate or inefficient. Where multiple surgeries are anticipated it is unnecessarily invasive to attach a closure device to the patient's sternum or rib cage.

It is critical that a sternal closure device remains stable to ensure a patient's sternum neither pulls apart, pushes together, or exhibits kinetic friction against each portion of the sternum. These forms of motion can scrape on vital organs, such as the heart, and damage the organ, leading to life-threatening conditions. Current sternal closure devices also do not have the ability to monitor sternal dehiscence or movement that requires medical attention.

Current devices and methods envision joining the two dissected portions of the sternum to allow the sternal bones to join together in the healing process. However, in situations where repeated access to the thoracic cavity may be necessary, it is inadvisable to allow the bones to join together.

Accordingly, there is a need for a system and method for longitudinally closing dissected sternums that is safe, reproducible, easy to perform, minimally invasive to the patient, possesses an internal sensor to monitor movement of the dissected sternum, and is designed to be readily removed for multiple procedures.

### BRIEF SUMMARY OF THE INVENTION

In situations where surgeons believe that repeated access to the patient's thoracic activity it is impractical to utilize a permanent or semi-permanent system and method of closing a patient's dissected sternum.

The invention is a temporary sternal closure and stabilization device for post-operatively closing, securing, and monitoring a patient's sternum following surgery resulting in a longitudinally severed sternum. The device consists of an open hook that wraps around the outside portion of the sternum and a cinching clip that stabilizes the device to the inside portion of the sternum. That hook and cinching clip then are connected over the dissected sternum by a cable, wire, or similar connector that is adjustable. On the other end of the connector is a corresponding hook and cinching clip that connects to the opposite end of the dissected sternum.

The cinching clips are removed by the surgeon if they are not necessary given the patient's medical need.

To place the device, a surgeon hooks one side of the device onto a patient's dissected sternum following the completion of a surgical procedure in the thoracic cavity. The surgeon then tightens the cinch clip using a specialized screwdriver or similar tightening device. The surgeon then affixes the second hook and cinch clip to the second portion of the dissected sternum.

The wire connecting both hooks is then tightened or crimped to achieve hemostasis in the patient. This is done using pliers. A series of these devices can be affixed to multiple points of the dissected sternum to achieve the desired stabilization of the sternum. The device is tightened sufficiently to stabilize the sternum but the two portions of the dissected sternum do not touch to avoid the possibility of the sternum bones from growing back together.

Alternatively, the device is deployed without the cinch clips. The cinch clips are removed if medically appropriate where only the hooks will provide sufficient stability to the patient's sternum.

The stress measurement dial detects motion or dehiscence of the sternum and alert medical providers of motion that may indicate dehiscence or lack of hemostasis in the patient's chest. The dial shows stress on the device. The dial also permits the surgeon to evaluate how much strength will be needed for definitive fixation following the final surgery.

The device is less invasive than existing devices in that the clamps do not wrap fully around the sternum. The device provides sufficient space within the thoracic cavity to insert medical grade paraffin gauze to shield internal vital organs, such as the heart, from rubbing against the sternum which causes injury to such organs, such as right ventricular rupture of the heart.

The device allows for patients that have undergone a sternotomy to have a stabilized sternum without the patient having to then have his/her chest medically paralyzed to avoid the use of such paralytic medications. The device is intended to be temporary and can be readily removed to allow for repeated surgeries in the thoracic cavity. The device intentionally leaves a small gap between the two portions of a dissected sternum to avoid the bones from growing back together in between surgical procedures. The device is reusable or disposable at the physician's discretion.

### BRIEF DESCRIPTION OF THE SEVERAL VIEWS OF THE DRAWING

FIG. 1 shows the anterior view of a dissected sternum where the device secures the two dissected portions between planned surgical procedures.

FIG. 2 shows the device consisting of opposing hooks joined with a wire or similar connection.

FIG. 3 shows the stress measurement dial that measures the pull force of the cinch clips on the sternum.

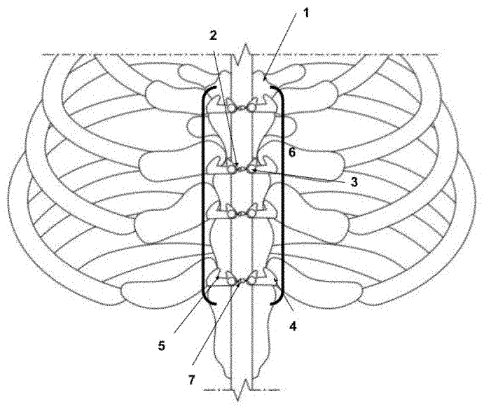

### DETAILED DESCRIPTION OF THE INVENTION

1. A sternum stabilization device for securing parts of a sternum 1 consisting of:
   a. A device 6 consisting of four sets of clamps consisting of a hook generally C-shaped leg for fixation onto the outer surface of one portion of a dissected sternum and a cinch clip 5 with a plurality of teeth that then apply opposing force on the interior surface of the same portion of the sternum to secure the device onto one portion of a dissected sternum;
   b. A wire that connects the sets of clamps 2;
   c. The cinch clip 5 tightens to the sternum with a screw device 3 (the cinch clip is removable and is removed from the device if not medically necessary by fully removing the screw at the junction of the hook and the cinch clip 3);
   d. The clamps have a general C-shaped portion 4;
2. The device comprises of a means of crimping or otherwise tightening a wire that pulls the two portions of a dissected sternum together following surgery in the thoracic cavity 2;
3. The device contains a stress measurement dial that provides a visual communication of the pull force on the sternum 7;
4. The device is readily removable using surgical scissors by cutting the wire 2 that holds the device in place;

1. A reusable sternal stabilization device comprising: Two corresponding open hooks; Two corresponding cinching clip; Cable wire connector; Stress measurement dial, wherein the improvement comprises a small gap between the two sides of the dissected sternum to allow for repeated access to the thoracic cavity.

2. A reusable medical device according to claim 1, configured to temporarily stabilize the two sides of a broken sternum.

3. A reusable medical device according to claim 1, configured to allow for the insertion of medical grade paraffin gauze.

4. A reusable medical device according to claim 1, designed to alert the physician of motion or dehiscence using it's stress measurement dial component.
